(19) United States
(12) Patent Application Publication
(10) Pub. No.: US 2018/0149630 A1
(43) Pub. Date: May 31, 2018

(54) SENSOR ELEMENT FOR DETECTING AT LEAST ONE PROPERTY OF A FLUID MEDIUM IN AT LEAST ONE MEASURING CHAMBER

(71) Applicant: Robert Bosch GmbH, Stuttgart (DE)

(72) Inventor: Uwe Konzelmann, Asperg (DE)

(21) Appl. No.: 15/822,771

(22) Filed: Nov. 27, 2017

(30) Foreign Application Priority Data
Nov. 30, 2016 (DE) 102016223834.5

(51) Int. Cl. G01N 33/00; H01M 8/0444

(52) U.S. Cl. CPC G01N 33/005; H01M 8/04462; G01N 33/0063

(57) ABSTRACT

A sensor element is provided for detecting at least one property of a fluid medium in at least one measuring chamber, in particular for detecting an H₂ proportion in a test gas. The sensor element comprises a reference chamber and a measuring diaphragm, the measuring diaphragm separating the reference chamber from the measuring chamber, the measuring diaphragm being at least partially heatable by at least one heating element. The sensor element furthermore has at least one channel, at least one fluid reference medium being applicable to the reference chamber through the channel.

### CROSS REFERENCE

The present application claims the benefit under 35 U.S.C. § 119 of German Patent No. 102016223834.5 filed on Nov. 30, 2016, which is expressly incorporated herein by reference in its entirety.

### BACKGROUND INFORMATION

A multitude of conventional sensor elements and methods detect at least one property of a fluid medium in a measuring chamber. This may fundamentally concern arbitrary properties of a gaseous or liquid fluid medium, it being possible to detect one or more properties. The present invention is described in the following, without limiting further specific embodiments and applications, in particular with reference to sensor elements for detecting a gas, in particular an H₂ proportion in a test gas.

Sensor elements of the kind described here are used in a multitude of areas, for example in automotive engineering, industrial processing engineering, chemistry and machine construction, in particular for determining gas concentrations. Thus, for example, the determination of hydrogen concentrations, for example in an air-hydrogen mixture, plays a large role in the application of hydrogen fuel cell systems. Safety-relevant applications should be mentioned in this connection as well. An air-hydrogen mixture becomes ignitable approximately at a hydrogen proportion of 4%. Sensor elements for detecting hydrogen may be used for example in hydrogen fuel cell vehicles in order, for example, to detect hydrogen escaping due to damage or defect and to trigger warning signals and/or protective measures by coupling to appropriate systems.

One principle for detecting a fluid medium in a test mixture of fluid media is based on the different thermal capacity and/or thermal conductivity of different fluid media, in particular of different components of a test mixture of fluid media, and is described for example in M. Arndt “Micromachined Thermal Conductivity Hydrogen Detector for Automotive Applications,” Sensors, 2002 IEEE. Thus, for example, hydrogen has a higher thermal conductivity than the gas components of air. A heatable measuring diaphragm may be in contact with the test mixture of fluid media. The heat emission of the diaphragm to the test mixture of fluid media may be determined for example via the measurement of the temperature of the heated measuring diaphragm or for example via a measurement of the heating power at a predefined temperature. Depending on the composition of the test mixture of fluid media, its thermal conduction changes and with appropriate calibration makes it possible to detect hydrogen in the test gas.

In spite of the advantages of the conventional sensor elements for detecting at least one property of a fluid medium, these still have room for improvement. In particular when conducting measurements at overpressure, for example at pressures above an atmosphere, the robustness of the sensor element plays a large role. In particular the measuring diaphragm is normally unable to withstand an overpressure and is often destroyed in measurements under overpressure.

### SUMMARY

The present invention provides a sensor element for detecting at least one property of a fluid medium in a measuring chamber, which at least largely avoids the disadvantages of known sensor elements for detecting at least one property of a fluid medium in a measuring chamber, and which in particular has a higher robustness against overpressure than the related art.

In the context of the present invention, a sensor element is fundamentally understood as any device which is able to detect the at least one property of the fluid medium and which is able to produce for example at least one measuring signal according to the detected property, for example an electrical measuring signal such as for example a voltage or a current. The property may be for example a physical and/or a chemical property. It is also possible to detect combinations of properties. The sensor element may be in particular designed to detect at least one property of a gas, in particular an H₂ proportion in a test gas. It is also possible to detect other properties and/or combinations of properties.

The sensor element may be designed in particular for use in a hydrogen fuel cell vehicle. The measuring chamber may be fundamentally any open or closed space, in which the fluid medium, in particular the test gas, is received and/or through which the fluid medium, in particular the test gas, flows.

The sensor element for detecting at least one property of a fluid medium in at least one measuring chamber, in particular for detecting an H₂ proportion in a test gas, includes a reference chamber and a measuring diaphragm. The measuring diaphragm separates the reference chamber from the measuring chamber and is at least partially heatable by at least one heating element. The sensor element furthermore has at least one channel. At least one fluid reference medium may be applied via the channel to the reference chamber.

In the context of the present invention, a reference chamber is fundamentally understood as any, open or closed, space, which is separated from the measuring chamber. In the context of the present invention, a measuring diaphragm is fundamentally understood as any separating layer, which is able to separate two spaces from one another and which is preferably designed in such a way, for example with respect to a material and/or a layer thickness, that an at least partial heating of a first side of the separating layer results in an at least partial warming of a second side of the separating layer. In the context of the present invention, a channel is fundamentally understood as any connection, for example a tubular passage, which connects two chambers to one another, in particular so as to enable an exchange of fluid media, in particular an exchange of gases, between the two chambers. In the context of the present invention, a fluid reference medium is fundamentally understood as an arbitrary fluid medium, which is preferably independent of the test gas and which has for example a known composition and/or a known property so that this reference medium may be used for a reference measurement. The reference medium in particular may be contained entirely or partially in the reference chamber.

The sensor element may be designed in particular to detect an H₂ proportion in a test gas, in particular a fuel gas in a fuel cell. The sensor element has at least one heating element and may furthermore have at least one temperature sensor. In the context of the present invention, a temperature sensor is fundamentally understood as any device that is designed to detect a temperature and to produce a measuring signal corresponding to the detected temperature. The heating element and the temperature sensor may be integrated in particular in a chip. The chip may be situated in particular in the reference chamber and may be in thermal contact with the measuring diaphragm. The chip may rest on the measuring diaphragm for example.

The sensor element may furthermore have a housing that encloses the reference chamber at least partially. In the context of the present invention, a housing is fundamentally understood as any component or a group of components, which enclose(s) the sensor element entirely or partially and/or close (s) the sensor element towards the outside and which are able to give the sensor element a mechanical stability. In particular, a housing may enclose at least one interior space. The housing may enclose the reference chamber, for example, at least partially and separate it off from its surroundings at least partially. The housing may be produced in particular entirely or partially from at least one of the following materials: a semiconductor material, a plastic, a metal. In particular, the measuring diaphragm is able to close off the reference chamber in a gas-tight manner from the measuring chamber. Furthermore, a side of the measuring diaphragm facing the reference chamber may have at least one protective element. In particular, the protective element may be selected from the group made up of: a glass or a ceramic coating. In the context of the present invention, a protective element is fundamentally understood as any element that is designed to protect the reference chamber and/or functional elements situated in the reference chamber, for example measuring elements and/or component situated in the reference chamber, particularly against corrosion, for example, by protecting against water, in particular against distilled water.

The sensor element may comprise at least one cap wafer and at least one measuring chamber wafer. In the context of the present invention, a cap wafer is to be understood as any wafer that bounds the reference chamber at least partially. In the context of the present invention, a measuring chamber wafer is to be understood fundamentally as any wafer that encloses the measuring chamber at least partially and/or separates it at least partially, especially from the reference chamber. The reference chamber may be formed in particular by the cap wafer and the measuring chamber wafer. The measuring chamber wafer may in particular comprise the measuring diaphragm. Furthermore, the measuring chamber wafer and the cap wafer may contain at least one material selected from the group made up of: silicon, silicon oxide, silicon nitride and silicon carbide. Furthermore, the cap wafer may comprise the channel at least partially. The channel may comprise a tube. Furthermore, the sensor element may comprise an electronics chamber. In particular, it is possible to apply the fluid reference medium to the electronics chamber. In particular, the channel may connect the reference chamber with the electronics chamber. In particular, as explained above, the at least one temperature sensor and the at least one heating element may be integrated in a chip. In this case, it is particularly favorable if the above-mentioned channel opens out into the reference chamber in such a way that a ventilation through the channel opens out directly above the chip.

The measuring diaphragm may furthermore have a central region. The at least one heating element is able to heat at least the central region of the measuring diaphragm at least partially. In the context of the present invention, a central region is fundamentally understood as any partial region of the measuring diaphragm that is in thermal contact with the at least one heating element. The sensor element may furthermore include additional functional elements, for example measuring elements and/or components such as, for example, at least one measuring resistor, at least one electrical contacting area and at least one conductor track. In particular, the measuring resistor, the electrical contacting area and the conductor track may be situated in the reference chamber.

In a further aspect of the present invention, a sensor system is provided for detecting at least one property of a fluid medium in at least one measuring chamber. The sensor system comprises at least one sensor element according to the present invention, that is, for example a sensor element according to one of the developments described above and/or according to one or more of the specific embodiments to be described in more detail below. The sensor system furthermore comprises at least one control unit, the control unit being designed to determine the at least one property using the sensor element. The control unit may be designed in particular to determine at least one thermal conductivity of the fluid medium using the sensor element. The control unit may furthermore be designed in particular to determine from the thermal conductivity at least a proportion of at least one component of the fluid medium, in particular a hydrogen proportion. The sensor system may furthermore comprise a pressure source. For this purpose, the pressure source may be fluidically connected to the channel and be designed to apply the fluid reference medium to the reference chamber. In this context, the fluid reference medium may have an overpressure. In the context of the present invention, an overpressure is fundamentally understood as a pressure above normal pressure. In particular, the overpressure may assume a value of above an atmosphere. The overpressure may have a value of 0 to 2 bar, for example, in particular between 0 and 2 bar above normal pressure. The pressure source may have a compressor, in particular an air compressor, and particularly preferentially a compressor of a fuel cell.

In another aspect of the present invention, a fuel cell system is provided. The fuel cell system comprises at least one fuel cell and at least one sensor system. The sensor system may be designed to detect at least a proportion of a gas component of a fuel gas in the fuel cell. The sensor system, however, may also be situated outside of the fuel cell and be designed to detect at least a proportion of a gas component of a fuel gas outside of the fuel cell.

In another aspect of the present invention, a method is provided for detecting at least one property of a fluid medium in at least one measuring chamber, in particular for detecting an H₂ proportion in a test gas. The method comprises a use of a sensor element, which comprises a reference chamber and a measuring chamber. The method furthermore comprises an at least partial heating of the measuring diaphragm by at least one heating element. The sensor element has a channel. The method comprises an application of at least one fluid reference medium to the reference chamber through the channel. The pressure of the fluid reference medium may essentially correspond to a pressure of the fluid medium, in particular of the test gas. In the context of the present invention, the term “essentially” is fundamentally understood to mean that two pressures do not differ by more than 40%, preferably by no more than 20%. The two pressures may in particular also be identical. In particular, the pressure of the fluid medium, in particular of the test gas, may be an overpressure. The overpressure may have a value of 0 to 1 bar, for example between 0 and 1 bar above normal pressure. Furthermore, the fluid reference medium may be a non-corrosive fluid reference medium, in particular a non-corrosive gas. In particular, the fluid reference medium may be an intake air following compression by an air compressor.

In another aspect of the present invention, a method is provided for producing a sensor element for detecting at least one property of a fluid medium in at least one measuring chamber. The sensor element comprises a reference chamber and a measuring diaphragm. The measuring diaphragm separates the reference chamber from the measuring chamber and is heatable by at least one heating element. The method comprises the following steps, preferably in the indicated order. Another order is fundamentally also possible. Furthermore, one or multiple or all of the method steps may also be performed repeatedly. Furthermore, one or more of the method steps may also be performed in an entirely or partially temporally overlapping manner or simultaneously. In addition to the mentioned method steps, the method may also comprise additional method steps.

The method steps include, for example:
a) providing at least one cap wafer and at least one measuring chamber wafer;
b) producing the measuring diaphragm by isotropic diaphragm etching of the measuring chamber wafer;
c) producing the measuring chamber by isotropic diaphragm etching of the cap wafer, at least one channel being produced in at least one wall of the cap wafer in the isotropic diaphragm etching of the cap wafer, at least one fluid reference medium being applicable to the reference chamber through the channel; and
d) mounting the cap wafer onto the measuring chamber wafer so that the measuring diaphragm separates the reference chamber from the measuring chamber.

The example device according to the present invention, the example operating method according to the present invention, and the example production method according to the present invention have numerous advantages vis-a-vis conventional devices, operating methods and production methods. In particular, the present invention makes it possible to increase the robustness of the sensor element, in particular the robustness of the measuring diaphragm, in particular with respect to an overpressure of the fluid medium, in particular of the test gas. In the event of an overpressure, in particular in the event of an operation under overpressure, it is possible that the fluid medium, in particular the test medium, which is under overpressure, is applied to the measuring diaphragm. On another side of the measuring diaphragm, the reference space is located, which normally is under atmospheric pressure. It is possible that the measuring diaphragm cannot withstand a differential pressure and is destroyed. By introducing a pressurized reference medium, in particular a gas, into the reference chamber, for example on a reference side, which is connected to the reference chamber via the channel, in particular via a vent, it is possible to reduce the differential pressure.

This may make it possible to increase the service life of the sensor element, in particular the service life of the measuring diaphragm, compared to the related art.

Furthermore, in a preferred specific embodiment of the present invention, the second fluid medium in a hydrogen fuel cell system may be taken from the intake air following compression by the air compressor. This makes it possible to build up a counterpressure in the reference chamber cost-effectively and using a non-corrosive medium. This makes it possible that by an application of the reference medium, functional elements such as, for example, heating elements, in particular a heater, measuring resistors and electrical contacting areas are not exposed to a corrosive medium and are protected against corrosion. Furthermore, it is possible to produce the channel, which may be situated in the cap wafer, together with the measuring diaphragm, which may be situated in the measuring wafer, in particular in one process step, for example in a process step using an isotropic diaphragm etching. This makes it possible to produce the sensor element in a cost-effective manner. It is possible to use the provided sensor element in versatile fashion. In particular, it is possible to use the sensor element as a safety sensor, for example for warning in the event of an escape of H₂. It is furthermore possible for the sensor element to be suitable as a process control sensor with detection of the H₂ concentration. It is furthermore possible for the sensor element to be used in hydrogen fuel cell vehicles.

### BRIEF DESCRIPTION OF THE DRAWINGS

Additional optional details and features of the present invention derive from the following description of preferred exemplary embodiments, which are shown schematically in the figures.

FIG. 1 shows a cross-sectional view of a sensor element of the present invention.

FIG. 2 shows a sectional view of a cap wafer used in FIG. 1.

FIG. 3 shows a schematic structure of a fuel cell system of the present invention.

### DETAILED DESCRIPTION OF EXAMPLE EMBODIMENTS

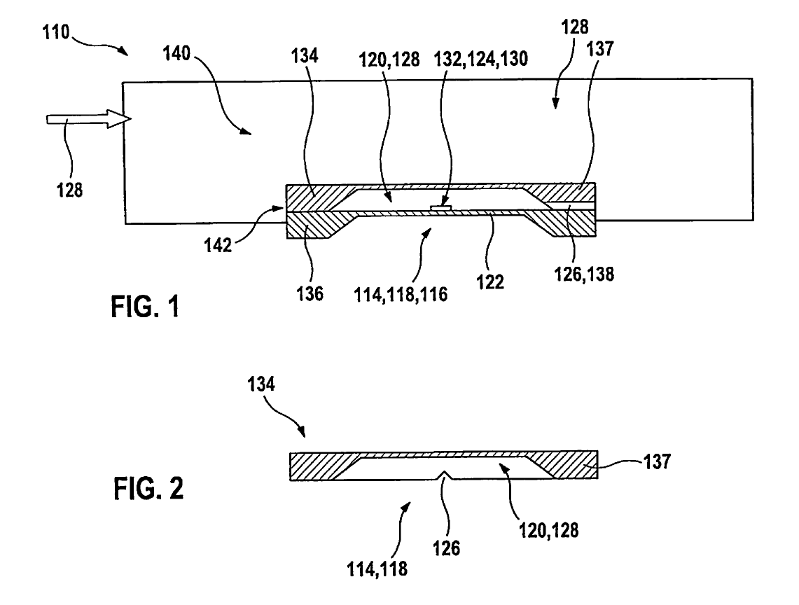

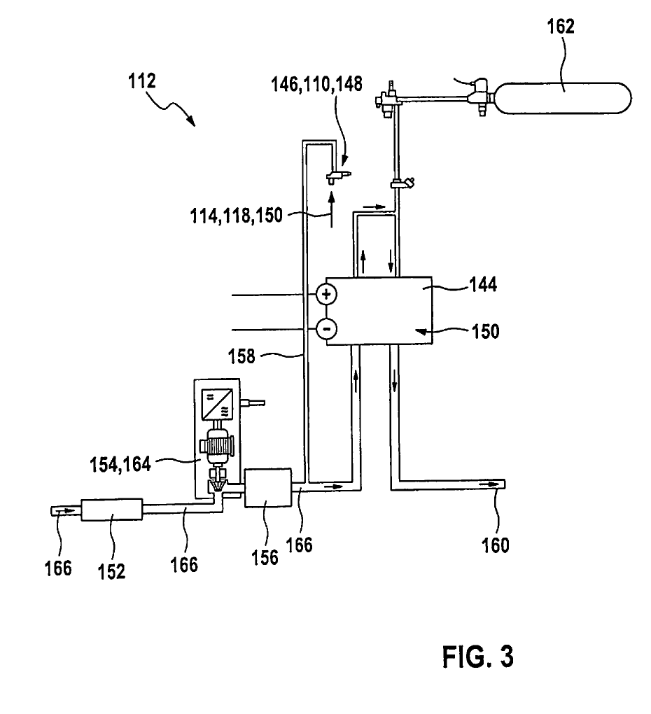

FIG. 1 shows a sensor element 110 according to the present invention in a cross-sectional view. FIG. 2 shows a cross section through a cap wafer 134 used in FIG. 1, the sectional plane running perpendicularly with respect to the sectional plane of FIG. 1. FIG. 3 shows an overview of a fuel cell system 112 according to the present invention. These figures are explained jointly below.

Sensor element 110 may be designed in particular for use in a hydrogen fuel cell vehicle. Other applications are also possible, however. Sensor element 110 may comprise in particular one or multiple additional functional elements, which are not shown in the Figures, such as for example electrodes, electrode leads and contacts, multiple layers or other elements, as shown for example in the above-mentioned related art.

Sensor element 110 for detecting at least one property of a fluid medium 114 in at least one measuring chamber 116, in particular for detecting an H₂ proportion in a test gas 118, comprises a reference chamber 120 and a measuring diaphragm 122. Measuring diaphragm 122 separates reference chamber 120 from measuring chamber 116 and is at least partially heatable by at least one heating element 124. Sensor element 110 furthermore has at least one channel 126. It is possible to apply at least one fluid reference medium 128 to reference chamber 120 through channel 126.

Sensor element 110 has at least one heating element 124 and may furthermore have at least one temperature sensor 130. Heating element 124 and temperature sensor 130 may be integrated in particular in a chip 132, as shown in FIG. 1, which may also be referred to as a sensor chip. As likewise shown in FIG. 1, chip 132 may be situated in reference chamber 120. Chip 132 may be in thermal contact with measuring diaphragm 122. Chip 132 for example may rest on measuring diaphragm 122, as seen in FIG. 1.

As likewise shown in FIG. 1, sensor element 110 may comprise at least one cap wafer 134 and at least one measuring chamber wafer 136. In particular, reference chamber 120 may be formed by cap wafer 134 and by measuring chamber wafer 136, as seen in FIG. 1. Measuring chamber wafer 136 may in particular comprise measuring diaphragm 122, as shown in FIG. 1. Measuring chamber wafer 136 and cap wafer 134 may furthermore have at least one material selected from the group made up of: silicon, silicon oxide, silicon nitride, silicon carbide. As shown in FIG. 1, cap wafer 134 may furthermore comprise channel 126 at least partially. Cap wafer 134 may have a wall 137. In particular, wall 137 of cap wafer 134 may encompass channel 126 at least partially. Channel 126 may comprise a tube 138.

To produce sensor element 110, it is possible to provide first cap wafer 134 and measuring chamber wafer 136. These may be respectively provided in the form of blanks. These blanks may then be processed, for example by an etching process, in which the blanks are etched simultaneously or also at different times. In this etching process, which may comprise for example an etching process using a wet chemical treatment and/or dry-etching, cavities may be respectively introduced into the blanks. Together with diaphragm 122, the cavity in cap wafer 134 may later form reference chamber 120. The cavity in measuring chamber wafer 136 may later form a part of measuring chamber 116. When forming the cavity of measuring chamber wafer 136, which may be achieved by isotropic diaphragm etching of measuring chamber wafer 136, diaphragm 122 may be formed, and at the same time channel 126 may be formed. Subsequently, cap wafer 134 may be mounted on measuring chamber wafer 136 so that measuring diaphragm 122 separates reference chamber 120 from measuring chamber 116.

Sensor element 110 may furthermore comprise an electronics chamber 140. In particular, it is possible to apply fluid reference medium 128 to electronics chamber 140. Fluid reference medium 128 may have an overpressure. In particular, channel 126 may connect reference chamber 120 to electronics chamber 140, as seen in FIG. 1.

Sensor element 110 may furthermore have a housing 142 at least partially enclosing reference chamber 120. As shown in FIG. 1, housing 142 may comprise cap wafer 134 and measuring chamber wafer 136. Measuring diaphragm 122 may in particular close off reference chamber 120 in a gas-tight manner vis-a-vis measuring chamber 116. Furthermore, a side of measuring diaphragm 122 facing reference chamber 120 may have at least one protective element that is not shown here. The protective element may be in particular selected from the group made up of: a glass and a ceramic coating. The measuring diaphragm may furthermore have a central region. At least the central region of measuring diaphragm 122 may be heatable at least partially by the at least one heating element 124. Other embodiments are also possible.

FIG. 3 shows a fuel cell system 112. Fuel cell system 112 comprises at least one fuel cell 144 and at least one sensor system 146. Sensor system 146 comprises at least one sensor element 110 and at least one control unit 148, control unit 148 being designed to determine the at least one property of the fluid medium 114 using sensor element 110. Control unit 146 may be designed in particular to determine at least one thermal conductivity of the fluid medium 114 using sensor element 110. Furthermore, control unit 146 may be designed in particular to determine from the thermal conductivity at least a proportion of at least one component of the fluid medium 114, in particular a hydrogen proportion. Sensor system 146 may be designed to detect at least a proportion of a gas component of a fuel gas 150 in fuel cell 144. As shown in FIG. 3, however, sensor system 146 may also be situated outside of fuel cell 144 and be designed to detect at least a proportion of a gas component of a fuel gas 150 outside of fuel cell 144.

FIG. 3 furthermore shows an air filter 152, an electric air compressor 154, an intercooler 156, a connection 158, an exhaust gas channel 160 and an H₂ tank 162. Sensor system 146 may comprise a pressure source 164. Pressure source 164 may be fluidically connected to channel 126 and be designed to apply the fluid reference medium 128, for example a non-corrosive gas, to reference chamber 120. The electric air compressor 154 is used in the exemplary embodiment described here as pressure source 164 of sensor system 146, an intake air 166 following compression by air compressor 154 acting as fluid reference medium 128. Electric air compressor 154 is here fluidically connected to channel 126 by connection 158. In the exemplary embodiment described here, in accordance with the method of the present invention, intake air 166, following compression by air compressor 154, is applied to reference chamber 120 through channel 126. For this purpose, a pressure of fluid reference medium 128, in particular of intake air 166 after compression by air compressor 154, may essentially correspond to a pressure of fluid medium 114, in particular of test gas 118, for example of fuel gas 150. The pressure of fluid medium 114, in particular of test gas 118, for example of fuel gas 150, may be an overpressure. Fluid reference medium 128, in particular intake air 166 after compression by air compressor 154, may have an overpressure. In particular, the overpressure may assume a value of above an atmosphere. The overpressure may exhibit a value from 0 to 2 bar, for example.

What is claimed is:

1. A sensor element for detecting at least one property of a fluid medium in at least one measuring chamber, the sensor element detecting an H₂ proportion in a test gas, the sensor element comprising:
a reference chamber;
a measuring diaphragm separating the reference chamber from the measuring chamber, the measuring diaphragm being at least partially heatable by at least one heating element; and
at least one channel, at least one fluid reference medium being able to be applied to the reference chamber through the channel.

2. The sensor element as recited in claim 1, wherein the sensor element is designed to detect an H₂ proportion in a test gas, the test gas being a fuel gas of a fuel cell.

3. The sensor element as recited in claim 1, wherein the sensor element includes the heating element and at least one temperature sensor.

4. The sensor element as recited in claim 1, wherein the sensor element includes a housing at least partially enclosing the reference chamber.

5. The sensor element as recited in claim 1, wherein a side of the measuring diaphragm facing the reference chamber has at least one protective element.

6. The sensor element as recited in claim 1, further comprising:
at least one cap wafer and at least one measuring chamber wafer, the measuring chamber wafer encompassing the measuring diaphragm, the cap wafer at least partially encompassing the channel.

7. A sensor system for detecting at least one property of a fluid medium in at least one measuring space, comprising:
at least one sensor element including a reference chamber, a measuring diaphragm separating the reference chamber from the measuring chamber, the measuring diaphragm being at least partially heatable by at least one heating element, and at least one channel, at least one fluid reference medium being able to be applied to the reference chamber through the channel; and
at least one control unit designed to determine the at least one property using the sensor element.

8. The sensor system as recited in claim 7, further comprising:
a pressure source fluidically connected to the channel and being designed to apply the fluid reference medium to the reference chamber, the fluid reference medium having an overpressure.

9. A fuel cell system, comprising:
at least one fuel cell; and
at least one sensor system for detecting at least one property of a fluid medium in at least one measuring space, including at least one sensor element including a reference chamber, a measuring diaphragm separating the reference chamber from the measuring chamber, the measuring diaphragm being at least partially heatable by at least one heating element, and at least one channel, at least one fluid reference medium being able to be applied to the reference chamber through the channel, and at least one control unit designed to determine the at least one property using the sensor element;
wherein the sensor element is designed to detect an H₂ proportion in a test gas, the test gas being a fuel gas of the fuel cell.

10. A method for detecting at least one property of a fluid medium in at least one measuring chamber, the method for detecting an H₂ proportion in a measuring gas, the method comprising:
providing a sensor element, the sensor element including a reference chamber, a measuring diaphragm and at least one channel;
at least partially heating the measuring diaphragm by at least one heating element, wherein the sensor element has a channel;
applying at least one fluid reference medium to the reference chamber through the channel.

11. A method for producing a sensor element for detecting at least one property of a fluid medium in at least one measuring chamber, the sensor element including a reference chamber and a measuring diaphragm, the measuring diaphragm separating the reference chamber from the measuring chamber, the measuring diaphragm being at least partially heatable by at least one heating element, the method comprising:
a) providing at least one cap wafer and at least one measuring chamber wafer;
b) producing the measuring diaphragm by isotropic diaphragm etching of the measuring chamber wafer;
c) producing the measuring chamber by isotropic diaphragm etching of the cap wafer, at least one channel being produced in at least one wall of the cap wafer in the isotropic diaphragm etching of the cap wafer, at least one fluid reference medium being able to be applied to the reference chamber through the channel; and
d) mounting the cap wafer on the measuring chamber wafer so that the measuring diaphragm separates the reference chamber from the measuring chamber.
